(19) United States
(12) Patent Application Publication
(10) Pub. No.: US 2024/0099296 A1
(43) Pub. Date: Mar. 28, 2024

(54) SYSTEM AND PROCESS FOR CONTINUOUS ADDITION OF CRYOPROTECTANT

(71) Applicant: ABS Global, Inc., DeForest, WI (US)

(72) Inventors: Domenic Busa, DeForest, WI (US); Michael Botts, DeForest, WI (US); Gary Klas, DeForest, WI (US); Johnathan Parker, DeForest, WI (US)

(21) Appl. No.: 18/527,805

(22) Filed: Dec. 4, 2023

Related U.S. Application Data
(63) Continuation of application No. 16/852,303, filed on Apr. 17, 2020, now Pat. No. 11,889,830.
(60) Provisional application No. 62/835,676, filed on Apr. 18, 2019.

(51) Int. Cl. A01N 1/02

(52) U.S. Cl. CPC A01N 1/0221; A01N 1/0242; A01N 1/0268

(57) ABSTRACT

An integrated system and method for preparing sperm cells to improve their survivability during cryopreservation are described herein. The system features a vessel, a controlled dispenser, and a dispense tube. The dispense tube has a first end fluidly connected to the dispenser and a second end disposed inside the vessel. The second end can be submerged in the sperm cell fluid. The controlled dispenser may be a syringe pump that includes a syringe for containing a cryoprotectant and a pushing mechanism for displacing the syringe. The syringe pump is configured to discharge the cryoprotectant through the dispense tube and into the vessel, thereby dispensing the cryoprotectant into the sperm cell fluid. Mixing of the fluids is achieved using a shaker table agitation.

### CROSS-REFERENCES TO RELATED APPLICATIONS

This application is a continuation and claims benefit of U.S. patent application Ser. No. 16/852,303, filed Apr. 17, 2020, which is a non-provisional and claims benefit of U.S. Patent Application No. 62/835,676, filed Apr. 18, 2019, the specifications of which are incorporated herein in their entirety by reference.

### BACKGROUND OF THE INVENTION

### Field of the Invention

The present invention relates in general to processing and cryopreservation of semen. In particular, the present invention discloses systems and method featuring high-precision and continuous addition of a fluid with agitation to optimize cryopreservation of sperm cells.

### Background Art

Cryopreservation of human spermatozoa involves the stabilization of sperm cells at cryogenic temperatures. This technology is commonly utilized in artificial insemination procedures or to preserve male fertility. During a cryopreservation procedure, a cryoprotectant is added to a sperm specimen to protect the sperm from freeze damage. A disadvantage of this procedure is that the uptake of the cryoprotectant imparts stress to the sperm cells, resulting in cell die-off. It is believed that a larger cryoprotectant to sperm cell concentration mismatch only increases this imparted stress. Past known solutions involve a drop-wise addition of cryoprotectant media via droplets breaking away from a suspended dispense tube, or step-wise addition comprising multiple discrete additions spaced minutes apart.

In drop-wise addition, a setup akin to a buret based titration is used. The cryo-protectant media (cryo-media) is added by drop over an extended period of time. In step-wise or bulk addition, the cryo-media is suspended in a vessel above the sperm sample. A stopcock attached to the vessel is used to start/stop flow. There is no deliberate control of the flow rate; instead, a mass balance is used to estimate the volume delivered. The addition of cryo-media is completed within minutes to read the desired total volume added. For example, the cryo-media may be added in three discrete bulk additions spaced 15 minutes apart. These procedures can still result in cell die-off; hence, there exists a need to reduce the rate of cell die off during the addition of a cryoprotectant media to sperm cells.

### BRIEF SUMMARY OF THE INVENTION

It is an objective of the present invention to provide systems and methods that can mitigate the detrimental effects of cryopreservation on sperm integrity, as specified in the independent claims. Embodiments of the invention are given in the dependent claims. Embodiments of the present invention can be freely combined with each other if they are not mutually exclusive.

In some aspects, the present invention features an integrated cryopreservation system that can improve survivability and process yield of sperm cells. In an exemplary embodiment, the present invention may feature a dispensing system comprising a vessel, a controlled dispenser, and a dispense tube. The controlled dispenser may be configured to dispense the fluid through the dispense tube and into the vessel. A non-limiting example of the controlled dispenser is a syringe pump comprising a syringe for containing a fluid, such as the cryoprotectant, and a pushing mechanism for displacing the syringe plunger. The syringe pump may be configured to dispense the fluid through the dispense tube and into the vessel upon displacement of the syringe by the pushing mechanism. In some embodiments, the dispense tube has a first end fluidly connected to a discharge port of the controlled dispenser and a second end disposed inside the vessel. In one embodiment, the second end may be contacting a material contained within the vessel. The material contained within the vessel may be cells, such as sperm cells, in a sample medium (i.e. a liquid media that contains e.g. salts, sugars, buffers, proteins, antibiotics, lipids, anti-oxidants, etc.). In the case of sperm cells, the sperm cells can be sexed (i.e. post-processing) or unsexed (i.e. “conventional”). The system may also be used for other cell types, e.g. cell cultures, oocytes, and embryos. Alternatively, or in conjunction, the second end may be contacting an internal surface of the vessel.

According to some embodiments, the pushing mechanism may comprise a moveable pusher block, a motor operatively coupled to said pusher block, and a motor controller for controlling the motor. The pusher block and syringe may be arranged on a track and aligned such that the pusher block can press against a plunger of the syringe. The motor is configured to move the pusher block along the track in a direction that pushes the plunger into a barrel of the syringe, thereby displacing the syringe. Displacement of the syringe ejects the fluid contained in the barrel through the discharge port and into the dispense tube. The fluid exits the dispense tube through the second end and into the vessel.

In other aspects, the dispensing system may further comprise a mixer for mixing the contents in the vessel. In further embodiments, the dispensing system may further comprise one or more additional controlled dispensers, and a dispense tube fluidly connecting each controlled dispenser to the vessel. The additional dispensers can dispense the same or different fluids, such as cryoprotectants or gases, through their respective dispense tubes and into the vessel upon displacement by the pushing mechanisms.

In one embodiment, the controlled dispenser can continuously dispense the fluid. In a non-limiting example, the pushing mechanism may displace the syringe plunger such that the fluid is dispensed continuously from the second end of the dispense tube that is submerged in the sperm cells. In another embodiment, the controlled dispenser can continuously dispense the fluid such that droplet formation is prevented and the fluid is dispensed without being dripped into the vessel. In yet other embodiments, the controlled dispenser can dispense the fluid into the vessel at a flow rate that is less than or equal to a maximum flow rate, fₘₐₓ, determined by the equation: fₘₐₓ (ml/min)=0.0045*V, where V=volume of material in ml. In alternative embodiments, (for example, embodiments with a shorter dispense time of around 45 minutes) the maximum flow rate, fₘₐₓ, may be determined by the equation: fₘₐₓ (ml/min)=0.0056*V, where V=volume of material in ml. The maximum flow rate is dictated by the volume of cryoprotectant (which is in turn dictated by the measured amount of sample), which is added over a set amount of time. Typically, the addition time is about 60 minutes, but in some instances may be about 45 minutes. For larger samples, the volume of cryoprotectant needed will be larger, and thus the maximum flow rate will be larger. Thus, the addition time may be roughly constant, regardless of the sample size and the volume of cryoprotectant added, because the total flow rate may be scaled linearly with the volume.

According to other embodiments, the cryopreservation system of the present invention may be utilized in a method for preparing the biological specimens for cryopreservation. When implementing the syringe pump as the controlled dispenser, the method may comprise adding the cryoprotectant into the syringe, adding the biological specimen into a vessel, connecting the syringe to the vessel via a dispense tube, and displacing the syringe to dispense the cryoprotectant into the vessel at a desired flow rate, thereby adding the cryoprotectant to the biological specimen. In one embodiment, one end of the dispense tube may be contacting the biological specimen or an internal surface of the vessel. In some embodiments, the cryoprotectant is continuously dispensed into the vessel. In a preferred embodiment, the syringe is displaced such that droplet formation is prevented and the cryoprotectant is dispensed without being dripped into the vessel. In other embodiments, the cryoprotectant is dispensed into the vessel at a flow rate that is less than or equal to the maximum flow rate, fₘₐₓ, determined by the previous equation.

One of the unique and technical features of the present invention is the continuous addition of the cryoprotectant to the specimen as compared to bulk or drop-wise addition. The same volume of cryoprotectant media, such as a glycerol-based cryoprotectant, can be added at a slow and continuous rate over a specified period of time. Another technical feature of the invention is the dispense tube submerged or contacting an internal surface of the vessel to enable low or soft impact addition of the cryoprotectant to the sperm cell solution. This feature can allow for gradual introduction of the cryoprotectant into the sperm cell solution in a manner that reduces cell stress. Yet another technical feature of the invention is the dilute addition of cryoprotectant in which the cryoprotectant enters the sperm cell fluid at a relatively low volume to reduce cytotoxity to local cells at or near the entry point. Without wishing to be limited by a particular theory or mechanism, it is believed that these technical features can reduce process risks and the rate of cell die off, thereby improving survivability and process yield of sperm cells. None of the presently known prior references or work has the unique inventive technical feature of the present invention.

In addition, the inventive technical feature of the present invention contributed to a surprising result. After extensive experimentation, it was unexpectedly found that through the cryopreservation preparation process, the survivability of cells increased by an average of 5% when using the submerged, continuous addition process in comparison to the step-wise addition process of three bulk additions spaced 15 minutes apart). The ratio of specimen volume to dispensed cryoprotectant volume was the same in both procedures. Furthermore, it was surprisingly found that cells prepared using the cryopreservation preparation process of the present invention had an 8% increase in survivability through the freeze/thaw procedure. Thus, the present invention was surprisingly able to increase the process yield of sperm cells, an outcome that one of ordinary skill in the art cannot simply predict or envision.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a non-limiting embodiment of a dispensing or cryopreservation system of the present invention.

FIGS. 2A-2B show a non-limiting example of a controlled dispenser of the dispensing system. FIG. 2A depicts a partially displaced syringe pump and FIG. 2B depicts the syringe pump in a completely displaced position.

FIG. 3 shows another non-limiting embodiment of the cryopreservation system.

FIG. 4A is another non-limiting embodiment of the cryopreservation system. FIG. 4B shows an embodiment where multiple specimens can be processed simultaneously.

FIG. 5A shows an initial position of a syringe clamp. FIG. 5B shows a displaced position of the syringe clamp holding a syringe.

FIG. 6A shows an initial position of an inductive position sensor that is connected to the syringe clamp; a change in position is proportional to a change in voltage. FIG. 6B shows a displaced position of the sensor when the syringe clamp is holding the syringe.

FIG. 7 is a non-limiting schematic of a pumping mechanism of the invention.

FIGS. 8A-8C are non-limiting examples of process flow diagrams for dispensing a cryoprotectant according to the present invention.

FIG. 9 shows non-limiting examples of feed streams for dispensing the cryoprotectant.

### DETAILED DESCRIPTION OF THE INVENTION

Following is a list of elements corresponding to a particular element referred to herein:

100 system

102 fluid or cryoprotectant

104 biological specimen

110 vessel

112 internal surface of the vessel

115 controlled dispenser, for example, a syringe pump

120 syringe

122 plunger

124 barrel

126 discharge port

127 syringe clamp

129 position sensor

130 pushing mechanism

132 pusher block

134 motor

136 motor controller

138 track

140 dispense tube

142 first end of dispense tube

144 second end of dispense tube

150 mixer

As used herein, a “cryoprotectant” refers to a chemical substance that is added to a biological specimen in order to protect said specimen from freeze damage during cryopreservation. The term “cryoprotectant” may be used interchangeably with the terms “cryoprotectant media” or “cryo-media”. Examples of cryoprotectants include, but are not limited to, glycerol, ethylene glycol, dimethyl sulphoxide (DMSO), sucrose, trehalose, dextrose, fructose, tris(hydroxymethyl)aminomethane (TRIS), TRIS B, Tris-Fructose-Egg yolk-Glycerol (TFEG), citric acid, and 1,2-propanediol.

As used herein, the “controlled dispenser” refers to any device capable of delivering a fluid, such as cryoprotectant or gas, at a controlled flow rate. In some preferred embodiments, the dispenser is automated. In other embodiments, the dispenser may be non-automated. Non-limiting examples of controlled dispensers that may be used in accordance with the present invention include a syringe pump, mechanical pump, peristaltic pump, gravity pump, direct lift pump, microfluidic pump, centrifugal pump, compressor, gas regulator, or any positive-displacement pump. In some preferred embodiments, the dispenser may be any flow controller that can deliver fluid with similar accuracy as a syringe pump. In another example, the controlled dispenser may comprise a vessel that has with it a controlled pressure above the fluid and a downstream flow rate measurement. A simple feedback loop controls pressure based on flow rate error to a set point. Alternatively, a throttling valve may be implemented to control flow instead of controlling pressure on the fluid.

As used herein, the term “syringe” refers to a positive-displacement pump comprising a barrel and a moveable, e.g. slidable, plunger that can be inserted into the barrel. The plunger can include a seal that allows for the plunger to create a suction when the plunger is pulled away from the barrel, thereby drawing and trapping fluids in the barrel. Pushing the plunger into the barrel forces, e.g. discharges, the trapped fluids from the barrel. As used herein, the term “displace” and derivatives thereof when used in conjunction with syringe refer to the plunger being pushed into the barrel.

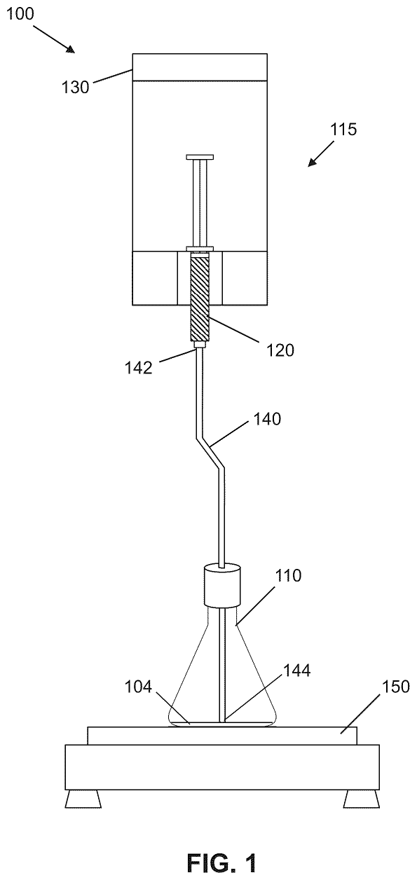

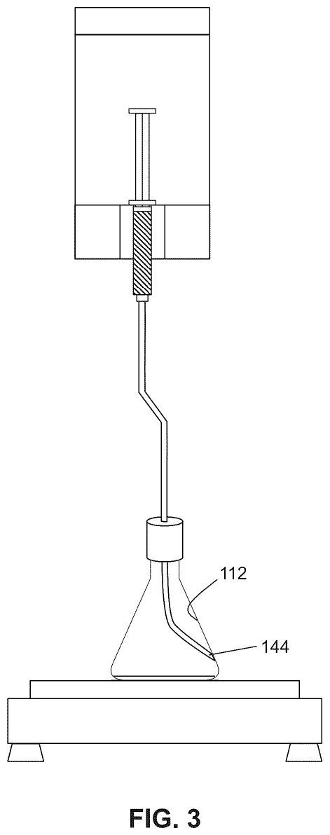

Referring now to FIG. 1, in one embodiment, the present invention features a dispensing system (100) comprising a vessel (110), a controlled dispenser (115), and a dispense tube. In some embodiments, the dispense tube (140) has a first end (142) fluidly connected to a discharge port (126) of the controlled dispenser and a second end (144) disposed inside the vessel (110). In one embodiment, the second end (144) may be contacting a material (104) contained within the vessel. In another embodiment, the second end (144) may be contacting an internal surface (112) of the vessel as shown in FIG. 3. Hereinafter, the controlled dispenser may be be exemplified by a syringe pump; however, it is to be understood that the controlled dispenser is not limited to a syringe pump and that it can be any device suited for delivering a fluid and controlling a flow rate thereof. In some embodiments, the syringe pump (115) may comprise a syringe (120) for containing a fluid (102) and a pushing mechanism (130) for displacing said syringe (120). The syringe pump (115) may be configured to dispense the fluid through the dispense tube (140) and into the vessel (110) upon displacement of the syringe (120) by the pushing mechanism (130).

According to some embodiments, the dispensing system (100) of the present invention may be utilized in cryopreservation procedure. For example, in one embodiment, the invention features a cryopreservation system (100) for dispensing a cryoprotectant (102) to a biological specimen (104). In some embodiments, the cryoprotectant (102) may comprise glycerol, ethylene glycol, dimethyl sulphoxide (DMSO), 1,2-propanediol, or any other suitable substance that can prevent damage to a biological specimen during freezing.

In one embodiment, the biological specimen (104) may comprise sperm cells. The sperm cells may comprise sex-sorted or gendered-skewed cells in which they predominantly contain an X-chromosome or a Y-chromosome. For example, the sperm cells may be sex-sorted to predominantly contain the X-chromosome so as to increase the likelihood that after insemination, the resulting fetus is a female. In another embodiment, the sperm cells may be unsorted. In other embodiments, the present invention is not limited to just sperm cells. Other types of specimens may be cryopreserved in accordance with the present invention. For example, the biological specimen may comprise tissues, stem cells, bone marrow, embryos, ova, oocytes, egg cells, amniotic fluid and umbilical cord, hepatocytes, blood cells, neuronal cells, organs, teeth, plant seeds, and microorganisms.

In one embodiment, the cryopreservation system (100) may comprise a vessel (110) for containing the biological specimen (104), a syringe pump (115) comprising a syringe (120) for containing the cryoprotectant (102) and a pushing mechanism (130) for displacing said syringe (120), and a dispense tube (140) having a first end (142) fluidly connected to a discharge port (126) of the syringe and a second end (144) disposed inside the vessel (110). The syringe pump (115) can dispense the cryoprotectant (102) through the dispense tube (140) and into the vessel (110) upon displacement of the syringe (120) by the pushing mechanism (130).

In some embodiments, the second end (144) may be in contact with an internal surface (112) of the vessel. For example, the second end (144) may be directly touching or positioned adjacent to the internal surface (112) of the vessel. In other embodiments, the second end (144) may be in contact with the biological specimen (104) when it is contained in the vessel (110). For instance, the second end (144) of the tube may be submerged in the biological specimen (104). In a preferred embodiment, the dispensed cryoprotectant (102) is prevented from being dripped into the vessel (110) by contacting the second end (144) of the tube with the biological specimen (104) or the internal surface (112) of the vessel. As used herein, the term “drip” and derivatives thereof refer to droplets breaking away from a suspended dispense tube.

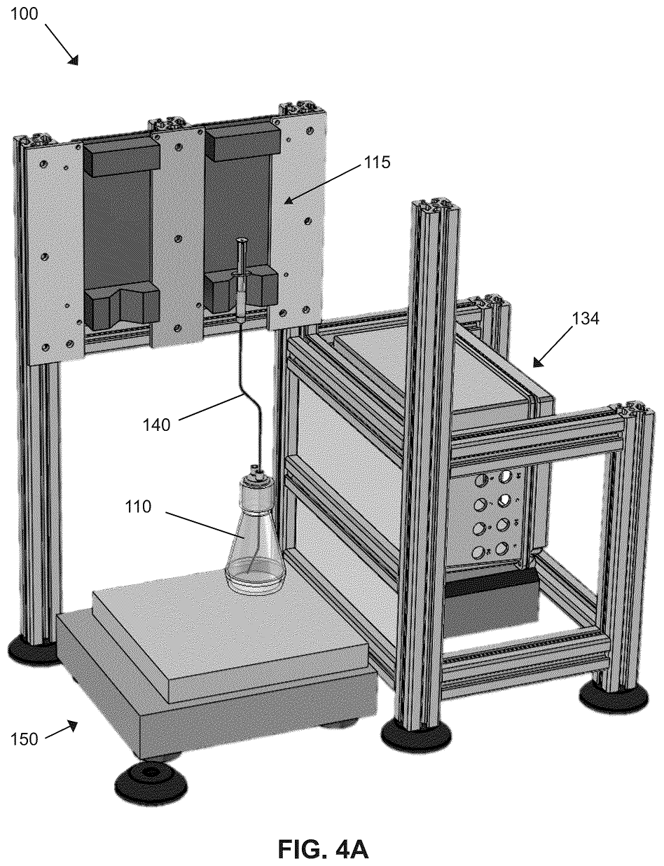

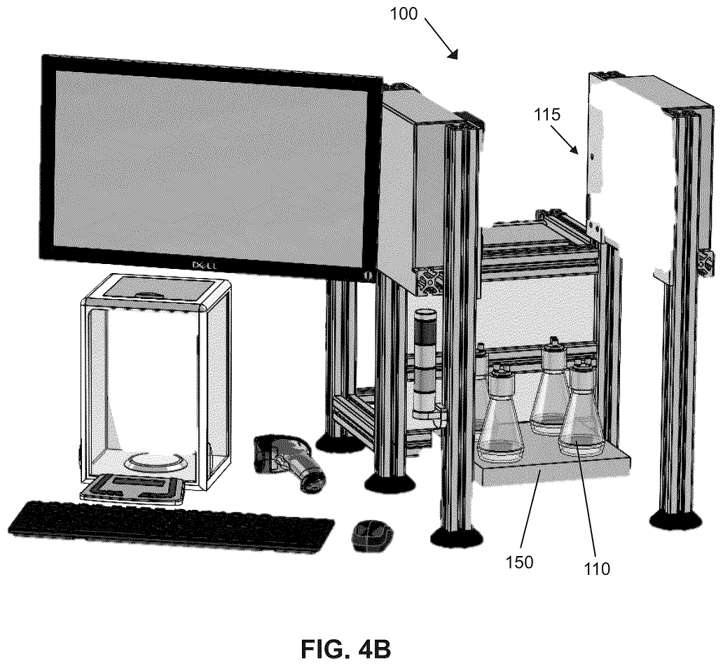

In one embodiment, the internal surface (112) of the vessel can include an internal side surface or internal bottom surface of the vessel. In some embodiments, the vessel (110) may be a flask, a tube, or any container having sufficient volumetric capacity to hold the cryoprotectant (102) and biological specimen (104). For example, the vessel (110) may have a volumetric capacity ranging from about 20 ml to about 100 ml. In another embodiment, the capacity of the vessel (110) may range about 100 ml to about 250 ml or about 250 ml to about 500 ml. In yet another embodiment, the capacity of the vessel (110) may be greater than 500 ml. In other embodiments, the vessel (110) may be an enclosed or sealed container. As shown in FIG. 4A, the vessel can include a cap that has an input port for receiving the dispense tube (140), thus the cap allows for the vessel to be sealed while still providing access to the interior of the vessel. In some embodiments, the vessel (110) may be constructed from a rigid or semi-rigid material, such as glass or plastic. In further embodiments, the material may be opaque if the specimen is light sensitive, or substantially transparent for visibility into the vessel.

In one embodiment, if the second end (144) of the dispense tube is in contact with the biological specimen (104), the cryoprotectant (102) is directly added to the biological specimen (104). For instance, the second end (144) is submerged in the biological specimen (104) and cryoprotectant (102) as the cryoprotectant (102) is added into the vessel. In another embodiment, if the second end (144) of the dispense tube is in contact with an internal surface of the vessel but not in contact with the biological specimen (104), e.g. the vessel side wall, the cryoprotectant (102) may be added to the biological specimen (104) indirectly. As shown in FIG. 3, if the second end (144) is touching the vessel side wall, the cryoprotectant (102) flows along the vessel side wall as it is gradually added to the specimen. In either embodiment, droplet formation is broken up and the dispensed cryoprotectant (102) does not drip into the vessel (110).

Without wishing to limit the present invention to a particular mechanism, the syringe pump can control the rate of delivery of the cryoprotectant by precise displacement of the syringe plunger. In one embodiment, the addition rate is set programmatically as a function of the volume of cells. Since the batch size of sperm cells can vary greatly based on the number of cells received from an ejaculate, a wide range of addition volumes must be satisfied. In some embodiments, the volume of sperm cell fluid may range from about 1 ml to about 100 mL per semen sample. In preferred embodiments, the total volume cryo-media added may be about 10% to about 25% of the sperm cell sample volume. For example, a 25 ml semen sample may require about 20% of cryo-media, thus the cryo-media volume is about 5 ml.

In some embodiments, the total cryo-media addition spans about 60 minutes for each batch. In other embodiments, the addition time may range from about 45 to 90 minutes. Since the total cryo-media volume changes as a function of the volume of sperm cell fluid, the flow rate is set based on target volume divided by the addition time. In one embodiment, the flow rate of the dispensed fluid, also referred to as addition rate, may range from about 0.01 ml/min to about 1 ml/min. For example, the flow rate may be 0.017-0.333 mL/min. In another embodiment, the flow rate may be about 0.01 ml/min to about 0.05 ml/min or about 0.05 ml/min to about 0.1 ml/min. In a further embodiment, the flow rate may be about 0.1 ml/min to about 1 ml/min or greater than 1 ml/min. Continuing with the previous example, 5 ml of cryo-media may be continuously added to the 25 ml semen sample in a span of about 60 minutes, thus the cryo-media flow rate is about 0.083 ml/min.

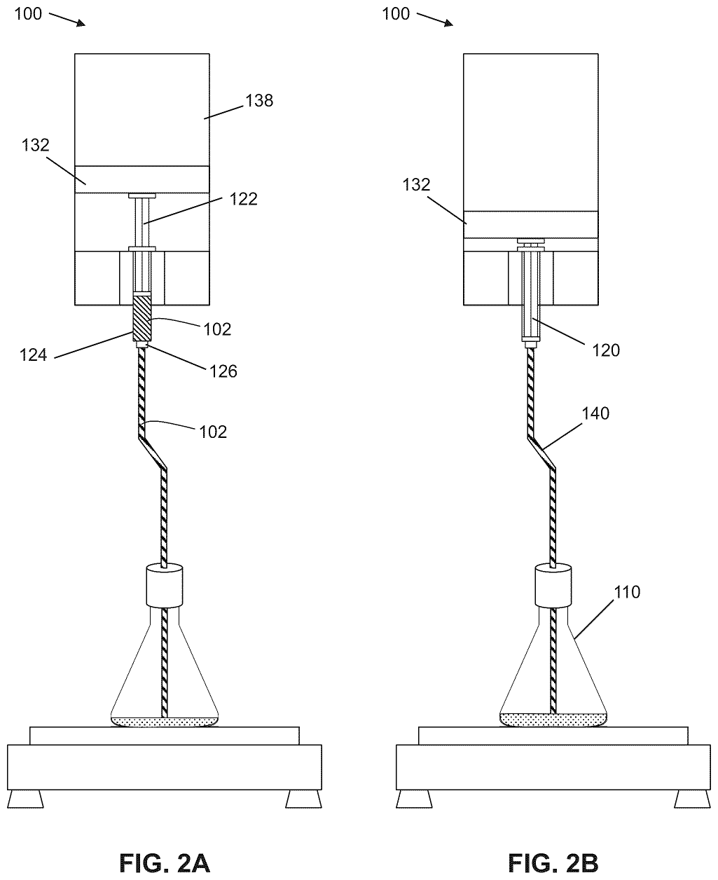

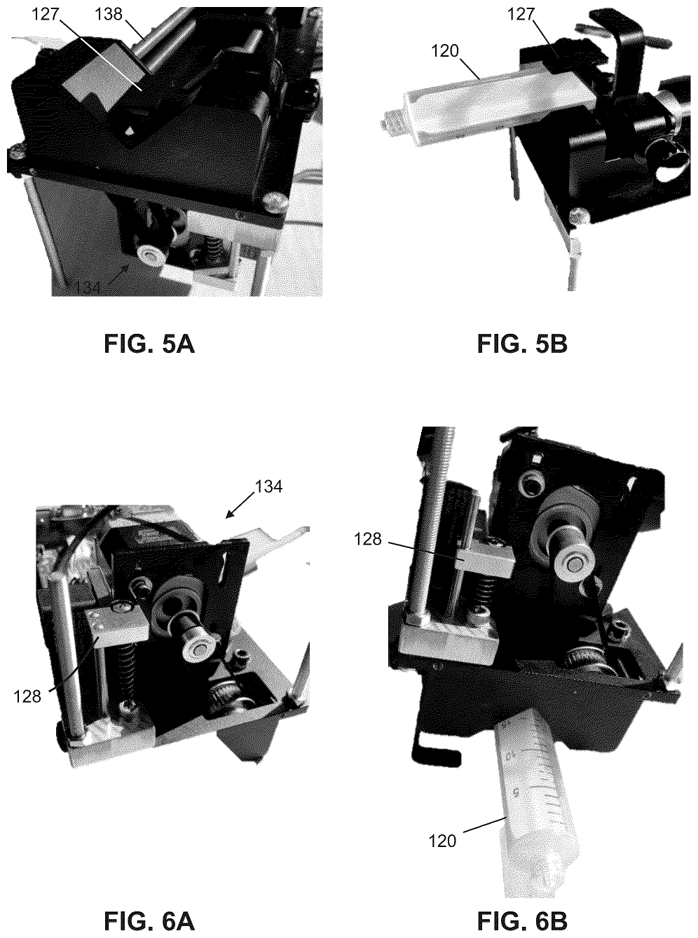

As shown in FIGS. 2A-2B, the pushing mechanism (130) may comprise a moveable pusher block (132) disposed on a track (138). The pusher block (132) and syringe (120) may be arranged on the track (138) such that the pusher block (132) is aligned with the syringe (120). The pusher block (132) can move along the track (138) to press against the plunger (122) of the syringe. Referring to FIG. 5A-7, the pushing mechanism (130) further includes a motor (134) operatively coupled to the pusher block (132), and a motor controller (136) for controlling the motor (134). The motor (134) may be configured to move the pusher block (132) along the track (138) in a direction that pushes the plunger (122) into the barrel (124) of the syringe, thereby displacing the syringe (120) to eject the fluid (102), such as the cryoprotectant, contained in the barrel (124) through the discharge port (126) and into the dispense tube (102). The fluid (102) then exits the dispense tube (140) through the second end (144) and into the vessel.

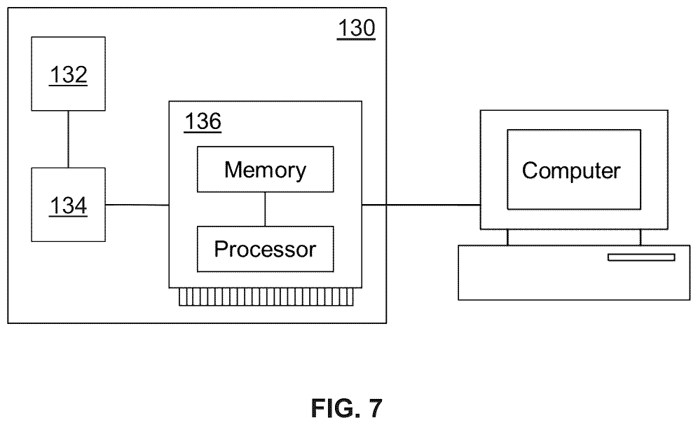

As shown in FIG. 7, in some embodiments, an external processor, such as a computer, is used to control the entire system. The computer can execute a LabVIEW software to operate the system. For instance, upon receiving a user-input which can be entered via peripheral devices such as a mouse or keyboard, the computer transmits signals to the motor controller, which may be integrated into the syringe pump. In one embodiment, the motor controller (136) may comprise a processor and a memory operatively coupled to the processor. The memory can store a set of instructions that, when executed by the processor upon receiving the computer signals, causes the motor (134) to move the pusher block (132) along the track at a desired step size in the direction to displace the syringe (120). Thus, the cryoprotectant (102) is added to the biological specimen (104) at a desired flow rate.

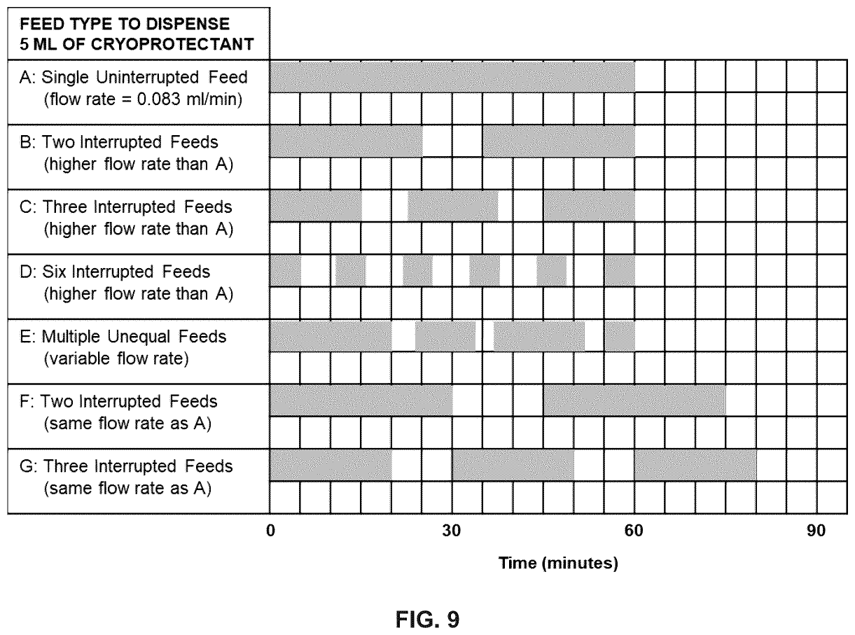

In some embodiments, a continuous flow may refer to a smooth or non-discrete flow, a steady flow or an uninterrupted flow. In one embodiment, the pusher block (132) displaces the syringe (120) such that the cryoprotectant (102) is added to the biological specimen (104) at a continuous flow rate. For example, the motor moves the pusher block (132) at a specific step size such that the cryo-media flow rate is about 0.083 ml/min to continuously dispense 5 ml of cryo-media into a 25 ml semen sample in about 60 minutes as shown in FIG. 9, Feed A.

In other embodiments, the pusher block (132) can displace the syringe (120) such that the cryoprotectant (102) is added to the biological specimen (104) at a pulsed flow rate or an interrupted flow rate. A pulsed or interrupted flow rate refers to a flow rate in which there are discrete breaks in the flow; however, the flow is not drop wise. For example, 25% of the cryo-media volume is slowly and continuously added to the specimen in a span of 10 minutes, then paused for 5 minutes, and then repeated until the total volume of cryo-media is dispensed. As another example, 10% of the cryo-media volume is slowly and continuously added to the specimen in a span of 3-4 minutes, then paused for 2-3 minutes, and then repeated until the total volume of cryo-media is dispensed. Various non-limiting examples of pulsed or interrupted flow are shown in FIG. 9, Feeds B-G. In some embodiments, the pulsed flow rate may be a steady or constant flow rate, such as in Feeds B-D and F-G. Alternatively, the cryoprotectant may be fed at a varying pulsed flow rate, e.g. non-constant flow rate, over the course of the stream as illustrated in Feed E. In preferred embodiments, the cryoprotectant is dispensed in a manner and/or flow rate that inhibits droplet formation.

In one embodiment, the accuracy in the addition may be a function of the syringe inside bore diameter, if the linear step size of the syringe pump is fixed. To achieve the same accuracy across a range of addition volumes, by percentage of delivered volume to target volume, multiple sized syringes can be used. In some embodiments, the control software specifies the size of syringe based on the volume of sperm cells. If the wrong syringe is placed on the system, the amount of cryoprotectant media delivered could be significantly greater or less than specified. This error amounts to significant loss of cells through either initial cell die off in this process (over-delivery) or increased die off measured after thawing of cryopreserved cells (under-delivery). This risk is mitigated by implementing an inductive position sensor (128) to determine an outside diameter of the syringe placed on the system. As shown in FIGS. 5A-6B, the inductive position sensor (128) may be connected to a syringe clamp (127). When the syringe clamp is holding the syringe, the sensor is displaced and this change in the position of the sensor is proportional to a change in voltage, which correlates to the outer diameter of the syringe.

In preferred embodiments, the syringe used in accordance with the present invention should be biocompatible with the specimen. The syringe should be free off lubricants or other substance that could be detrimental to sperm cells. A non-limiting example of a syringe that is suitable for use with the present invention is a NORM-JECT luer lock syringe. In some embodiments, the volumetric capacity of the syringe may range from about 2 ml to about 50 ml. In one embodiment, the volumetric capacity of the syringe may be about 2 ml to about 5 ml. In another embodiment, the volumetric capacity of the syringe may be about 5 ml to about 20 ml. In yet another embodiment, the volumetric capacity of the syringe may be about 20 ml to about 50 ml. In further embodiments, the volumetric capacity of the syringe may be greater than 50 ml.

In other embodiments, the dispense tube may be constructed from a plastic, rubber, or silicone material. For example, the dispense tube may be made of PVC, DEHP-Free PVC, Tygon®, C-flex®, high density polyethylene, platinum cured silicone, or peroxide cured silicone. Moreover, the tubing material may be medical grade quality. In one embodiment, the dispense tube may be constructed of PEEK with 1/16″ OD, 0.020″ ID. An inner diameter of the tube can vary in size, ranging from about 0.01″ to about 0.1″. For example, the inner diameter may be 0.020″. The dispense tube can be a predetermined length or alternatively cut to a required length to sufficiently span the distance between the syringe and vessel. In some embodiments, the tubing material may be opaque. Alternatively, the tubing material may be substantially transparent to allow for visibility into the tube, for instance, to visualize any blockage in the tube. In other embodiments, the dispense tube may be flexible, semi-rigid, or rigid. Tubing expansion under pressure may increase error to the target delivery volume. To ensure accuracy, the tube can have a high OD/ID ratio with a rigid material, thereby reducing the risk of tubing expansion.

In the prior arts, a suspended vessel with a stopcock or a buret for addition has physical limitations that prohibits submersion of the dispense tip. For instance, the typical opening size of the vessel or the size of buret tip limits the ability to submerge the tip. In addition, by using a buret, which is typically made of brittle glass, physical contact with a vessel wall would place a bending force on the glass putting it at risk for fracture. Radial agitation of the vessel would only increase the fracture risk. In contrast, the dispense tube of the present invention allows for submersion of the dispense tip as well as agitation of the vessel.

Once sperm cells are packaged for sale, each lot is tested for bacteria. If bacteria levels are above a threshold, the batch is not sold as it could introduce a health risk. Furthermore, prevention of cell cross-contamination between batches is paramount. Thus, there is a need to prevent the cryo-media from becoming contaminated with bacteria and/or other cells. The present invention addresses these concerns by using a reusable or disposable fluid path. Without wishing to be limited to a particular theory or mechanism, the entire fluid path can eliminate contamination risks from bacteria and cross-contamination between different specimens, e.g. bull-bull.

In some embodiments, the dispense tubing and vessel may be reused through a cleaning and autoclave process. In other embodiments, the syringe may be discarded after use. In preferred embodiments, the dispense tubing is submerged into the fluid containing the sperm cells. By submerging the end of the dispense tubing instead of suspending it, the addition rate is governed by the syringe pump rather than the adhesion forces of suspending a droplet from the end of the dispense tubing.

In further embodiments, the system (100) may also include a mixer (150) for mixing the cryoprotectant (102) and the biological specimen (104) contained in the vessel (110). Without wishing to limit the present invention, the mixer (150) can prevent the biological specimen (104) from adhering to the internal surface (112) of the vessel. In one embodiment, the mixer (150) may be a shaker table. As an example, the shaker table may comprise a stationary base and a moveable platform that can support the vessel. As shown in FIG. 4A, the vessel (110) may be placed on the shaker table, which provides agitation to the fluid in the vessel, thereby mixing the cryoprotectant media into the fluid containing the sperm cells and reducing the local concentration at the end of the dispense tubing. The agitation rate of the shaker table may be programmatically set by rotational speed if the rotation radius is fixed by the equipment. While the shaker table can reduce contamination by eliminating contact with the contents of the vessel, other types of mixer equipment may be used with the system, such as a magnetic stir bar with stir plate or an overhead stirrer with an impeller, paddle, or blade. However, since there is a need to minimize shear forces on the sperm cells, which may be physically destroyed by a submerged rotating object, acoustic mixing may be implemented in accordance with the present invention. For example, acoustic mixing may involve sonication, which is agitation using sound energy.

As shown in FIG. 4B, in some embodiments, the dispense system (100) may be a multiple dispense system implementing a plurality of controlled dispensers and vessels to process multiple specimens simultaneously. In some embodiments, each vessel may contain its own specimen, which can be the same as or different from the specimens in the other vessels. Each vessel may be coupled to its own controlled dispenser, such as a syringe pump for example, to dispense its own a fluid. The fluid in each dispenser may be the same as or different from the fluid in the other dispensers. For example, one dispense system may be used to dispense glycerol into a semen sample and a second dispense system may be used to dispense glycerol into another semen sample of the same or different origin. As another example, one dispense system may be used to dispense glycerol into a tissue sample while a second dispense system may be used to dispense glycerol into a semen sample. In another embodiment, one dispense system may be used to dispense glycerol into an embryo specimen while a second dispense system may be used to dispense DMSO into a semen sample. The vessels can be placed on a single shaker table provided that the shaker table is capable of supporting and mixing a plurality of vessels. Alternatively, each vessel may have its own mixer.

In other embodiments, the multiple dispense system may implement a plurality of controlled dispensers, such as syringe pumps, fluidly coupled to a single vessel. This may allow for a shorter processing time by dispensing the cryoprotectant fluid from multiple dispensers. Alternatively, the dispensers can dispense various fluids, such as cryoprotectants or gases, into the single vessel simultaneously or sequentially. For instance, the dispense system may include multiple syringe pumps that dispense various fluids into a single vessel for vitrification of sperm cells. Each of the fluids can be dispensed at their own independent flow rates and timing. In one embodiment, the dispense system may include a syringe pump that dispenses a cryoprotectant and a second controlled dispenser that dispenses gas into a single vessel. Each of the fluids can be dispensed at their own independent flow rates and timing. In one example, the multiple dispense system may be configured to dispense two or more different medias that are added to the cells such that delivery is defined similar to a recipe. The flow rates of each media may be varied independently and/or the delivery between the medias may be coordinated, such as ramping the flow rate.

In some embodiments, the vessel may be equipped with a cap that can create an air-tight seal such that a gas can be added and contained within the vessel. The cap may include a port for receiving the gas, as well as other ports for receiving liquids. As an example, preparation of a specimen for in-vitro fertilization may require lower oxygen concentrations and/or some type of gas coverage in the vessel to improve survivability. In one embodiment, the vessel may be sealed and purged with argon, CO2 and/or nitrogen, which may be added via one or more ports, each coupled to its own pump or gas regulator. A separate exit port may be used to maintain atmospheric conditions and prevent pressure build-up in the vessel. In another embodiment, the process may operate under reduced pressure by coupling a port to a vacuum pump. In some embodiments, the cryo-media can be added with the gas via a separate input port and pump. In conjunction with or alternative to the syringe pumps, other types of pumps, such as a microfluidic pump, centrifugal pump, compressor, gas regulator, or another kind of positive-displacement pump, may be used in accordance with the present invention to dispense the cryo-media, gases, or other fluids.

According to some embodiments, the dispense systems of the present invention may implement a temperature ramp during the cryopreservation process. When preparing the biological specimen for cryopreservation, the specimen and cryoprotectant are preferably in a temperature controlled environment. In a preferred embodiment, the specimen and cryoprotectant are maintained at a temperature of about 5° C. Any deviation in temperature should not exceed ±2° C. For example, the temperature deviation may be ±1° C. In some embodiments, the entire system may be located in a temperature controlled room. If a dedicated cold room is not available, at least the syringe pump and vessel may be contained in a temperature controlled chamber, such as a cold box, so as to maintain constant temperature of the specimen and cryoprotectant. In another embodiment, the vessel and/or syringe may be jacketed with a heating/cooling device. Temperature feedback may be implemented via a thermocouple or thermistor, which can be submerged in the specimen solution or attached to the vessel.

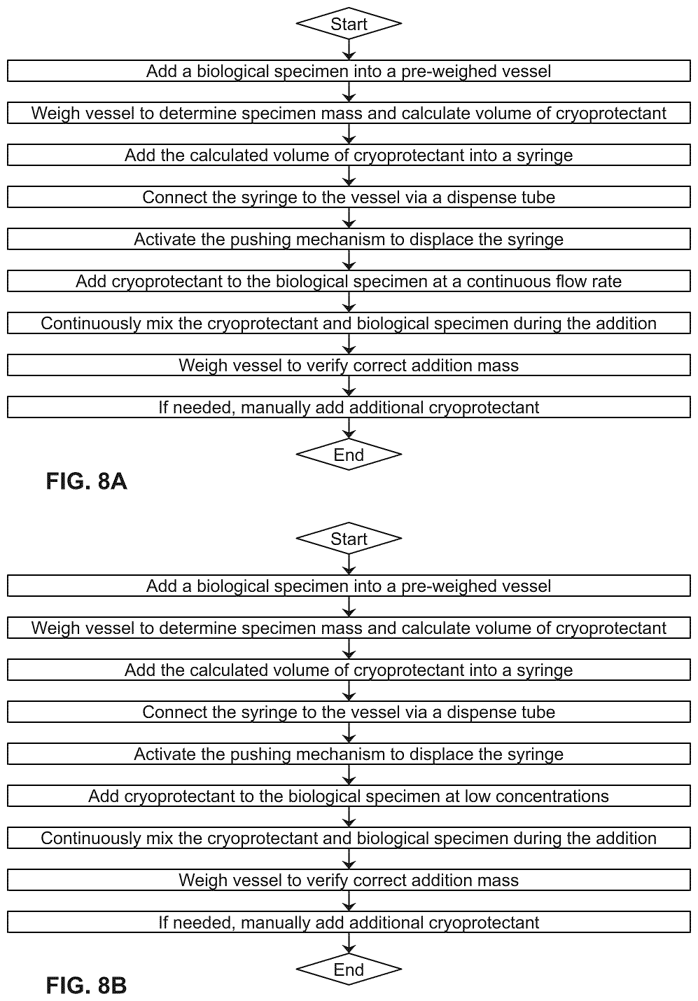

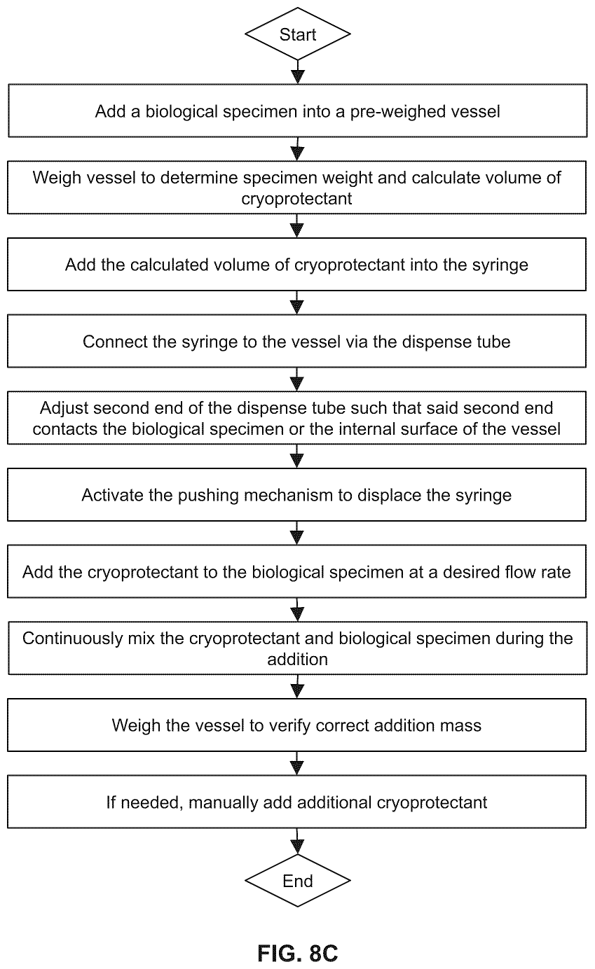

According to some aspects, the systems described herein may be utilized in methods for dispensing a cryoprotectant to a biological specimen as shown in FIGS. 8A-8C. As a key initial step, the volume of cryoprotectant to be added may be determined, based on the amount of the sample, by pre-weighing the vessel, adding the sample, weighing the combined vessel and sample, and subtracting the masses to determine the mass of the sample, and calculating the proper volume of cryoprotectant. In one embodiment as shown in FIG. 8A, the method may comprise providing any one of the cryopreservation systems (100) described herein, fluidly coupling the cryoprotectant (102) to the controlled dispenser (115), adding the biological specimen (104) into the vessel (110), and activating the controlled dispenser (115) to continuously dispense the cryoprotectant (102) through the dispense tube (140) and into the vessel (110). In one embodiment, the controlled dispenser (115) comprises a syringe pump having a syringe and pumping mechanism as previously described herein. Preferably, the pushing mechanism (130) displaces the syringe (120) such that the cryoprotectant (102) is added to the biological specimen (104) continuously and at a desired flow rate. This can prevent droplet formation so that the cryoprotectant (102) is dispensed without being dripped into the vessel (110).

In another embodiment as shown in FIG. 8B, the method may comprise providing any one of the cryopreservation systems (100) described herein, adding a desired volume of cryoprotectant (102) into the syringe (120), adding the biological specimen (104) into the vessel (110), and activating the pushing mechanism (130) to displace the syringe (120). By displacing the syringe, the cryoprotectant (102) is dispensed through the dispense tube (140) and into the vessel (110). Preferably, the pushing mechanism (130) displaces the syringe (120) such that the cryoprotectant (102) is added to the biological specimen (104) at a flow rate that is less than or equal to a maximum flow rate, fₘₐₓ, determined by the equation:

fₘₐₓ (ml/min)=0.0045*V,

where V=volume of the biological specimen in ml.
Returning to the previous example, the fₘₐₓ for 5 ml of cryoprotectant to be dispensed into the 25 ml semen sample is calculated to be 0.11 ml/min. Thus, the cryoprotectant flow rate should be less than or equal to 0.11 ml/min with a minimum dispense time of about 45 minutes. The previous example flow rate of 0.083 ml/min and dispense time of 60 minutes are within these parameters.

Referring now to FIG. 8C, another embodiment of the method may comprise providing any one of the cryopreservation systems (100) described herein, adding a desired volume of cryoprotectant (102) into the syringe (120), adding the biological specimen (104) into the vessel (110), adjusting the second end (144) of the dispense tube such that said second end (144) is contacting the biological specimen (104) or an internal surface (112) of the vessel, and activating the pushing mechanism (130) to displace the syringe (120). By displacing the syringe, the cryoprotectant (102) is dispensed through the dispense tube (140) and into the vessel (110). Preferably, the pushing mechanism (130) displaces the syringe (120) such that the cryoprotectant (102) is added to the biological specimen (104) at a desired flow rate. More preferably, by contacting the second end (144) of the dispense tube with the internal surface (112) of the vessel or the biological specimen (104), the dispensed cryoprotectant (102) is prevented from being dripped into the vessel (110).

Consistent with the previous embodiments, the methods described herein may further include continuously or intermittently mixing the cryoprotectant (102) and the biological specimen (104) contained in the vessel (110). The cryoprotectant (102) and the biological specimen (104) may be mixed using any of the mixing equipment described herein. In further embodiments, the methods may also comprise adding one or more additional fluids to the vessel via activation of one or more additional controlled dispensers that are fluidly connected to the vessel, each dispenser containing one of the additional fluids. This method may utilize the multiple dispense system as previously described. In some embodiments, the one or more additional fluids may be cryoprotectants or gases. For example, a second fluid may be added to the vessel via activation of a second syringe pump which has a syringe containing the second fluid and is fluidly connected to the vessel.

Consistent with the previous embodiments, the methods described herein may further include weighing the vessel at the end of the addition to verify that the correct mass of cryoprotectant has been added. At this stage, if the mass is determined to be lower than it should be, additional cryoprotectant may be manually or automatically added in order to bring the total amount of cryoprotectant to the calculated amount. If the volume of the secondary, or manual, addition is large in comparison to the total calculated volume, it may be added in multiple sub-volume additions, with waiting time intervals between them. As a non-limiting example, if the volume of manual addition is up to 32% of the total volume the full remaining volume may be delivered in one addition. If the volume of the manual addition is 33-65% of the total volume, it may be divided into two equal quantities which are added with a 15 minute break between the additions. If the volume of the manual addition is equal or greater to 66% of the total volume, it may be divided into three equal quantities which are added with two 15 minute breaks between the additions.

In some aspects, the cryoprotectant may be toxic to the biological specimen, such as sperm cells, in high concentrations. Dispensing the cryoprotectant via bulk or drop-wise addition can deliver immediate high concentrations of the cryoprotectant to the cells that are closest to the entry location of the cryoprotectant prior to being diffused in the cell solution, thus resulting in cytotoxicity to the local cells. In other aspects, drop-wise addition of the cryoprotectant can lead to stress or physical damage due to the droplets striking the cells. Without wishing to limit the present invention to a particular theory or mechanism, the present invention allows for addition of the cryoprotectant in smaller volumes, via slow continuous flow, low-impact entry, and/or a determined flow rate, that reduce the local concentration so as to minimize cytotoxicity and cell stress.

In some embodiments, the dispensing system of the present invention may be used in a cold room or a large cold handling cabinet, so as to preserve sample viability. In an alternative embodiment, the system may be fit inside of a fridge or mini-fridge. Some components of the system, such as the frame, may need to be decreased in size so that the frame, main syringe pumps, and shaker table all fit inside the fridge or mini-fridge. In some embodiments, a secondary frame may be positioned outside of the fridge or mini-fridge (for example, on top of the fridge or mini-fridge) and components such as the computer, power supply, and monitor may be mounted to the secondary frame.

In some embodiments, the pump plunger may be designed so as to also act as a spring-loaded clamping piece that holds the syringes into the pump and block. Certain plungers and clamps may cause inconsistencies of measurement. As a non-limiting example, the sensor to tag distance of the unit may be altered, and may in turn cause the system to incorrectly identify what syringe was loaded into the pump. As such, specialty plungers may be advantageous. Additionally, specialty syringe pump covers may be advantageous so as to provide for safer cable management and easier maintenance removal.

### EXAMPLE

The following is a non-limiting example of the present invention. It is to be understood that said example is not intended to limit the present invention in any way. Equivalents or substitutes are within the scope of the present invention.

The cryopreservation system was implemented and compared against a prior cryopreservation procedure. The cryoprotectant was added by the present system at a continuously flow rate, whereas the prior procedure involved three discrete additions spaced 15 minutes apart. The ratio of specimen volume to dispensed cryoprotectant volume was the same in both procedures. The present invention resulted in a 5% recovery of cells, thus the present invention provides, on average, 5% more cells than the prior procedure. Without wishing to limit the present invention to a particular theory or mechanism, the cryopreservation system reduced cell die off through cryopreservation freeze to thaw. Furthermore, the present invention resulted in an 8% increase in insemination dose in straws as compared to the prior procedure.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for preparing a biological specimen (104) for cryopreservation, said method comprising:
a) providing a cryopreservation system (100) comprising:
i. a vessel (110);
ii. a syringe pump (115) comprising a syringe (120) and a pushing mechanism (130) for displacing said syringe (120); and
iii. a dispense tube (140) having a first end (142) fluidly connected to a discharge port (126) of the syringe and a second end (144) disposed inside the vessel (110);
b) adding a desired volume of a cryoprotectant (102) into the syringe (120);
c) adding the biological specimen (104) into the vessel (110); and
d) activating the pushing mechanism (130) to displace the syringe (120), thereby dispensing the cryoprotectant (102) through the dispense tube (140) and into the vessel (110), wherein the pushing mechanism (130) displaces the syringe (120) such that the cryoprotectant (102) is continuously added to the biological specimen (104) at a desired flow rate and droplet formation is prevented, wherein the cryoprotectant (102) is dispensed without being dripped into the vessel (110).

2. The method of claim 1, wherein the pushing mechanism (130) comprises a moveable pusher block (132), a motor (134) operatively coupled to said pusher block (132), and a motor controller (136) for controlling the motor (134),
wherein the pusher block (132) and syringe (120) are arranged on a track (138) and aligned such that the pusher block (132) can press against a plunger (122) of the syringe, wherein the motor (134) is activated by the motor controller (136) to move the pusher block (132) along the track (138) in a direction that pushes the plunger (122) into a barrel (124) of the syringe, thereby displacing the syringe (120).

3. The method of claim 1 further comprising mixing the cryoprotectant (102) and the biological specimen (104) contained in the vessel (110).

4. The method of claim 1 further comprising adding another fluid to the vessel via activation of a second syringe pump that is fluidly connected to the vessel, wherein the second syringe pump includes a syringe containing the other fluid.

5. A method for preparing a biological specimen (104) for cryopreservation, said method comprising:
a) providing a cryopreservation system (100) comprising:
i. a vessel (110);
ii. a syringe pump (115) comprising a syringe (120) and a pushing mechanism (130) for displacing said syringe (120); and
iii. a dispense tube (140) having a first end (142) fluidly connected to a discharge port (126) of the syringe and a second end (144) disposed inside the vessel (110);
b) adding a desired volume of a cryoprotectant (102) into the syringe (120);
c) adding the biological specimen (104) into the vessel (110);
d) adjusting the second end (144) of the dispense tube such that said second end (144) is contacting the biological specimen (104) or an internal surface (112) of the vessel; and
e) activating the pushing mechanism (130) to displace the syringe (120), thereby dispensing the cryoprotectant (102) through the dispense tube (140) and into the vessel (110), wherein the pushing mechanism (130) displaces the syringe (120) such that the cryoprotectant (102) is added to the biological specimen (104) at a desired flow rate;
wherein by contacting the second end (144) of the tube with the internal surface (112) of the vessel or the biological specimen (104), droplet formation is prevented and the cryoprotectant (102) is dispensed without being dripped into the vessel (110).

6. The method of claim 5, wherein the pushing mechanism (130) comprises a moveable pusher block (132), a motor (134) operatively coupled to said pusher block (132), and a motor controller (136) for controlling the motor (134),
wherein the pusher block (132) and syringe (120) are arranged on a track (138) and aligned such that the pusher block (132) can press against a plunger (122) of the syringe, wherein the motor (134) is activated by the motor controller (136) to move the pusher block (132) along the track (138) in a direction that pushes the plunger (122) into a barrel (124) of the syringe, thereby displacing the syringe (120).

7. The method of claim 5, further comprising mixing the cryoprotectant (102) and the biological specimen (104) contained in the vessel (110).

8. The method of claim 5, further comprising adding another fluid to the vessel via activation of a second syringe pump that is fluidly connected to the vessel, wherein the second syringe pump includes a syringe containing the other fluid.

9. A cryopreservation system (100) for dispensing a cryoprotectant (102) to a biological specimen (104), said system (100) comprising:
a) a vessel (110) for containing the biological specimen (104);
b) a syringe pump (115) comprising a syringe (120) for containing the cryoprotectant (102), and a pushing mechanism (130) for displacing said syringe (120); and
c) a dispense tube (140) having a first end (142) fluidly connected to a discharge port (126) of the syringe and a second end (144) disposed inside the vessel (110) such that said second end (144) is contacting an internal surface (112) of the vessel or the biological specimen (104) when it is contained in the vessel (110);
wherein the syringe pump (115) is configured to dispense the cryoprotectant (102) through the dispense tube (140) and into the vessel (110) upon displacement of the syringe (120) by the pushing mechanism (130),
wherein by contacting the second end (144) of the tube with the internal surface (112) of the vessel or the biological specimen (104), droplet formation is prevented and the cryoprotectant (102) is dispensed without being dripped into the vessel (110).

10. The system of claim 9, wherein the second end (144) of the tube is submerged in the biological specimen (104).

11. The system of claim 9, wherein the pushing mechanism (130) comprises a moveable pusher block (132), a motor (134) operatively coupled to said pusher block (132), and a motor controller (136) for controlling the motor (134).

12. The system of claim 11, wherein the pusher block (132) and syringe (120) are arranged on a track (138), wherein the pusher block (132) is aligned with the syringe (120) such that the pusher block (132) can press against a plunger (122) of the syringe, wherein the motor (134) is configured to move the pusher block (132) along the track (138) in a direction that pushes the plunger (122) into a barrel (124) of the syringe, thereby displacing the syringe (120),
wherein displacement of the syringe (120) ejects the cryoprotectant (102) contained in the barrel (124) through the discharge port (126) and into the dispense tube (102), wherein the cryoprotectant (102) exits the dispense tube (140) through the second end (144) and into the vessel (110).

13. The system of claim 11, wherein the motor controller (136) comprises a processor, and a memory operatively coupled to the processor and storing a set of instructions that, when executed by the processor, causes the motor (134) to move the pusher block (132) at a desired step size in the direction to displace the syringe (120) such that the cryoprotectant (102) is added to the biological specimen (104) at a desired flow rate.

14. The system of claim 13, wherein the pushecryr block (132) displaces the syringe (120) such that the cryoprotectant (102) is added to the biological specimen (104) at a continuous flow rate.

15. The system of claim 13, wherein the pusher block (132) displaces the syringe (120) such that the cryoprotectant (102) is added to the biological specimen (104) at a pulsed flow rate.

16. The system of claim 9, further comprising a mixer (150) for mixing the cryoprotectant (102) and the biological specimen (104) contained in the vessel (110), wherein the mixer (150) prevents the biological specimen (104) from adhering to the internal surface (112) of the vessel.

17. The system of claim 16, wherein the mixer (150) is a shaker table, wherein the vessel (110) is placed on the shaker table, wherein shaking of the shaker table agitates the cryoprotectant (102) and the biological specimen (104).

18. The system of claim 9, further comprising one or more additional syringe pumps (115), each comprising a syringe (120) containing another fluid and a pushing mechanism (130) for displacing said syringe (120), and a dispense tube (140) fluidly connecting each syringe (120) to the vessel (110), wherein the one or more additional syringe pumps (115) are configured to dispense the other fluid through the dispense tube (140) and into the vessel (110) upon displacement of the syringe (120) by the pushing mechanism (130).

19. The system of claim 9, wherein the cryoprotectant (102) comprises glycerol, ethylene glycol, dimethyl sulphoxide (DMSO), or 1,2-propanediol.

20. The system of claim 9, wherein the biological specimen (104) comprises sperm cells.
